(19) United States
(12) Patent Application Publication
(10) Pub. No.: US 2025/0152424 A1
(43) Pub. Date: May 15, 2025

(54) EAR CORRECTION APPARATUS

(71) Applicants: Ji Hee Kim, Seoul (KR); Seo Hyung Ahn, Seoul (KR)

(72) Inventors: Ji Hee Kim, Seoul (KR); Seo Hyung Ahn, Seoul (KR)

(21) Appl. No.: 18/949,675

(22) Filed: Nov. 15, 2024

(30) Foreign Application Priority Data
Nov. 15, 2023 (KR) 10-2023-0158618

(51) Int. Cl. A61F 11/00

(52) U.S. Cl. CPC A61F 11/00

(57) ABSTRACT

Provided is an ear correction apparatus capable of effectively correcting the shape of an ear.

### CROSS-REFERENCE TO RELATED APPLICATION

This application is based on and claims priority under 35 U.S.C. § 119 to Korean Patent Application No. 10-2023-0158618, filed on Nov. 15, 2023, in the Korean Intellectual Property Office, the disclosure of which is incorporated by reference herein in its entirety.

### BACKGROUND

### 1. Field

The disclosure relates to an ear correction apparatus, and more particularly, to an ear correction apparatus capable of effectively correcting the shape of the ear.

### 2. Description of the Related Art

Ear deformities or malformations are common in newborns. About 30% of these ear deformities or malformations improve on their own, but for the remaining cases, children will have to live with ear deformities or malformations until they are 4 to 5 years old when surgery becomes possible.

However, surgery carries risks such as anesthesia, inflammation, and postoperative complications, and also may incur high costs. Therefore, to correct ear deformities non-surgically, it is desirable to use a correction apparatus within four weeks of birth when the ear cartilage is still flexible.

Thus, there is a need for an apparatus capable of correcting ear deformities in a non-surgical manner.

### PRIOR ART DOCUMENTS

### Patent Documents

Korean Patent No. 2125389

### SUMMARY

Provided is an ear correction apparatus capable of effectively correcting the shape of an ear.

Additional aspects will be set forth in part in the description which follows and, in part, will be apparent from the description, or may be learned by practice of the presented embodiments of the disclosure.

According to an aspect of the disclosure, an ear correction apparatus includes a core having rigidity, ductility, and deformability properties, and extending a predetermined length, a first correction portion connected to the core, and a second correction portion connected to the core at a position away from the first correction portion, wherein the first correction portion includes a support line having a predetermined curvature, the second correction portion includes a plurality of correction balls, the plurality of correction balls are arranged in a row to form a first array line, the core penetrates the first correction portion in a direction parallel to the support line of the first correction portion, and the core penetrates the plurality of correction balls along the first array line.

According to an embodiment, the core may include a first line portion penetrating the first correction portion and a second line portion penetrating the second correction portion, wherein at least a portion of the first line portion and at least a portion of the second line portion may extend parallel to each other at a predetermined distance from each other, the support line of the first correction portion and the second correction portion may face each other, and a correction space having a predetermined width may be formed between the support line of the first correction portion and the second correction portion.

According to an embodiment, the plurality of correction balls of the second correction portion may have different sizes from each other.

According to an embodiment, the plurality of correction balls of the second correction unit may include first correction balls and second correction balls having a diameter less than a diameter of the first correction balls, the first correction balls may be positioned at a center portion of the first array line, and the second correction balls may be positioned at end portions of the first array line.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a view illustrating an ear correction apparatus according to an embodiment;

FIG. 2 is a view illustrating a first correction portion in a separated state;

FIG. 3 is a view illustrating body parts around the ear on which the ear correction apparatus will be worn according to an embodiment;

FIG. 4 is a view illustrating how the ear correction apparatus is worn according to an embodiment;

FIG. 5 is a view illustrating the ear correction apparatus worn by a user according to an embodiment; and

FIG. 6 is a view illustrating operational effects of the ear correction apparatus according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, embodiments will be described with reference to the accompanying drawings.

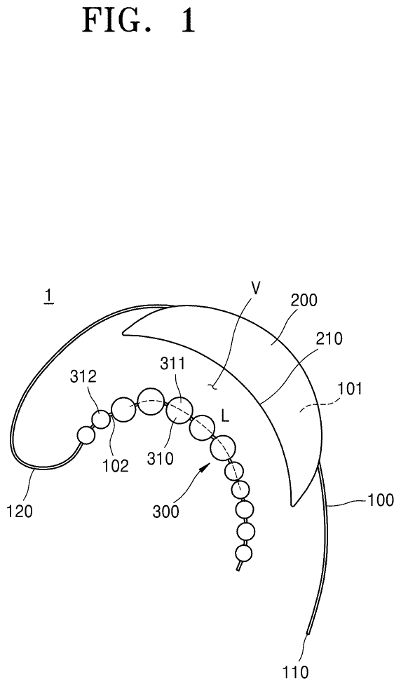

FIG. 1 is a view illustrating an ear correction apparatus 1 according to an embodiment.

According to an embodiment, the ear correction apparatus 1 may be used for correcting the shape of an ear, for example, the shape of an ear of a newborn.

According to an embodiment, the ear correction apparatus 1 may include a core 100 having rigidity, ductility, and deformability properties and extending for a predetermined length, a first correction portion 200 connected to the core 100, and a second correction portion 300 connected to the core 100 at a position away from the first correction portion 200.

### <Core 100>

The core 100 may be a wire with a predetermined length and thickness. The length and thickness of the core 100 are not limited and may be determined such that the core 100 may connect the first correction portion 200 and the second correction portion 300 to each other and provide an ear correction effect when being worn on the ear of a user.

The core 100 may deform when an external force exceeding a certain threshold is applied to the core 100 and may maintain its shape when an external force less than the threshold is applied to the core 100. The core 100 may include a material such as iron, a synthetic resin, or stainless steel. For example, the core 100 may be configured to not affect the body even when being brought into contact with the body. For example, the core 100 may include a material harmless to the human body or may be surface treated. Alternatively, according to an embodiment, the core 100 may include a member surrounding the surface of the core 100. In addition, core end portions 110 that are end portions the core 100 in a length direction of the core 100 may be processed into a rounded shape or the like. Alternatively, protective members may be provided on the core end portions 110. Thus, even when the core end portions 110 touch the body, the body may not be damaged by the core end portions 110.

The core 100 may include a first line portion 101 and a second line portion 102. The first line portion 101 and the second line portion 102 may not be separate members but may be part of the core 100. In other words, the first line portion 101 and the second line portion 102 may be continuous with each other.

The first line portion 101 that is part of the core 100 may penetrate the first correction portion 200, and the first correction portion 200 may be fixed to the first line portion 101. For example, the first line portion 101 may be adjacent to one of the core end portions 110 of the core 100.

The second line portion 102 that is part of the core 100 may penetrate the second correction portion 300, and the second correction portion 300 may be fixed to the second line portion 102. For example, the second line portion 102 may be adjacent to the other core end portion 110 of the core 100.

At least a portion of the first line portion 101 and at least a portion of the second line portion 102 may extend parallel to each other at a predetermined distance from each other. That is, as shown in FIG. 1, the first line portion 101 and the second line portion 102 may each extend with a certain curvature, and a portion of the first line portion 101 and a portion of the second line portion 102 may extend parallel to each other at a predetermined distance from each other. Here, the term “parallel” is used to indicate not only perfectly parallel extension but also extension with a certain angle therebetween. That is, the term “parallel” may be broadly interpreted to include not strictly parallel portions.

In this case, a curved portion 120 may be provided between the first line portion 101 and the second line portion 102. The curved portion 120 may be a portion rounded with a predetermined radius of curvature. The first line portion 101 and the second line portion 102 are connected to each other through the curved portion 120 such that the first line portion 101 and the second line portion 102 may extend parallel to each other.

### <First Correction Portion 200>

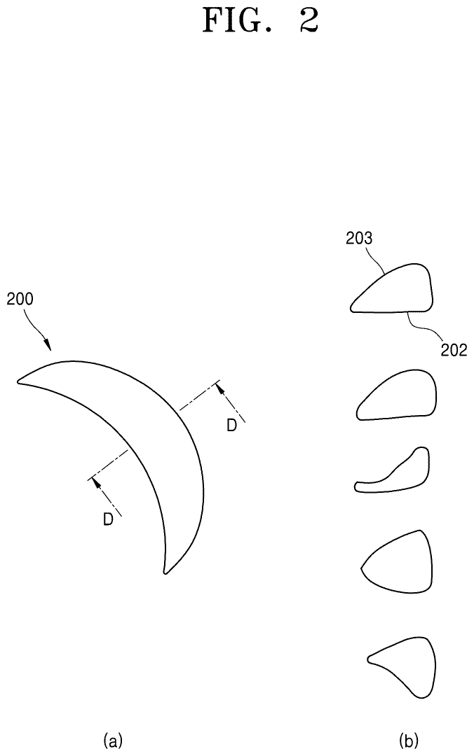

FIG. 2 is a view illustrating the first correction portion 200 in a separated state. In FIG. 2, section (a) shows an overall shape of the first correction portion 200, and section (b) shows various cross-sectional shapes along line D-D of section (a) according to embodiments.

The first correction portion 200 may be a plate-like member with a predetermined size and area. Here, the term “plate” does not necessarily refer to a plate of uniform thickness throughout, and may refer to a plate with a partially non-uniform thickness. The first correction portion 200 may be inserted into a space between the wearer's ear and head and may be fixed in position within the space. Additionally, the first correction portion 200 may have a size and shape for such positional fixation.

For example, as shown in section (b) of FIG. 2, the first correction portion 200 may generally have a triangular cross-sectional shape or a curved cross-sectional shape. The first correction portion 200 may include an inner surface 202 and an outer surface 203, and the inner surface 202 and the outer surface 203 may have a sloped or curved surface.

The material of the first correction portion 200 is not limited. For example, the first correction portion 200 may include a lightweight material that is capable of elastic deformation when subjected to external force and is harmless to the body when brought into contact with the body. For example, the first correction portion 200 may include a synthetic resin that is harmless to the human body.

The first correction portion 200 may include a support line 210.

The support line 210 may be part of an edge of the first correction portion 200. The support line 210 may extend with a predetermined curvature. The support line 210 may have a curvature and extension length for making contact with and being supported by a body line between the wearer's ear and head. Therefore, the first correction portion 200 may be inserted and fixed in position within the space between the wearer's ear and head.

In an embodiment, the first correction portion 200 may have a crescent shape. The support line 210 may be an inner curved portion (having a large radius of curvature) of the crescent shape of the first correction portion 200.

As described above, the core 100 includes the first line portion 101, and the first line portion 101 may penetrate the first correction portion 200. In this case, the direction in which the first line portion 101 penetrates the first correction portion 200 may be parallel to the support line 210. In other words, the first line portion 101 and the support line 210 may extend parallel to each other. Here, as described above, the term “parallel” is used to indicate not only perfectly parallel extension but also extension with a certain angle therebetween. That is, the first line portion 101 and the support line 210 may not be strictly parallel to each other in some portions.

### <Second Correction Portion 300>

The second correction portion 300 may include a plurality of correction balls 310. In other words, the second correction portion 300 may include a set of multiple correction balls 310.

The material of the second correction portion 300 and the correction balls 310 forming the second correction portion 300 is not limited. For example, the second correction portion 300 may include a lightweight material that is capable of elastic deformation when subjected to external force and is harmless to the body when brought into contact with the body. For example, the second correction portion 300 may include a synthetic resin that is harmless to the human body.

The correction balls 310 may be arranged in a line along a first array line L. The first array line L is not a physical line but rather a virtual line formed by the alignment of the correction balls 310.

As described above, the core 100 includes the second line portion 102, and the second line portion 102 may penetrate the second correction portion 300. In this case, the second line portion 102 may extend along the first array line L. In other words, the correction balls 310 may be connected together by the core 100 penetrating the correction balls 310 and may be arranged in a line by the core 100 along the first array line L.

A correction space V may be formed between at least a portion of the first correction portion 200 and at least a portion of the second correction portion 300.

For example, the support line 210 of the first correction portion 200 and the second correction portion 300 may face each other. Thus, a space with a certain width may be formed as the correction space V between the support line 210 of the first correction portion 200 and the second correction portion 300.

In an embodiment, the correction balls 310 forming the second correction portion 300 may have different sizes.

In an embodiment, the correction balls 310 forming the second correction portion 300 may include first correction balls 311 and second correction balls 312.

The first correction balls 311 may have a predetermined diameter. The second correction balls 312 may have a diameter less than the diameter of the first correction balls 311.

In an embodiment, the first correction balls 311 may be provided at a center portion of the first array line L. In addition, the second correction balls 312 may be provided at end portions of the first array line L.

In an embodiment, the diameter of the first correction balls 311 may be two to three times the diameter of the second correction balls 312. However, this is merely an example, and embodiments are not limited thereto.

### <Uses and Effects of Ear Correction Apparatus 1 of Disclosure>

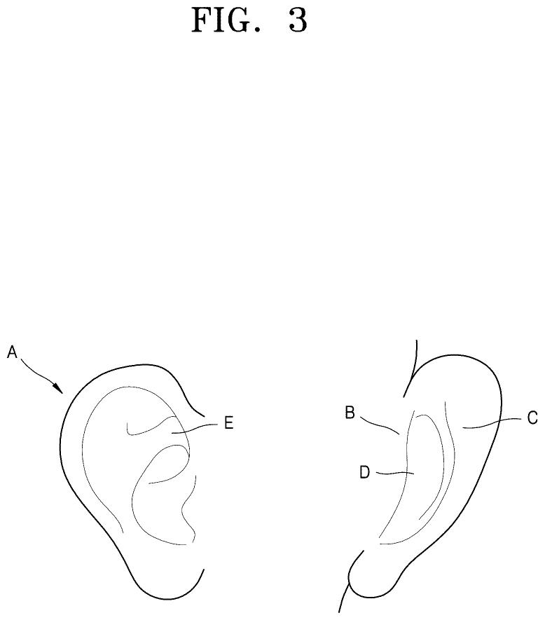

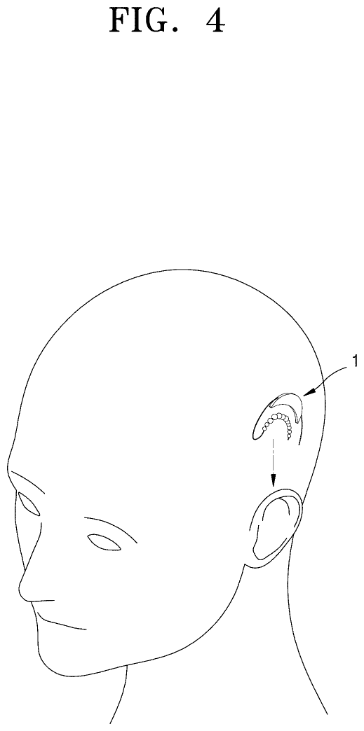

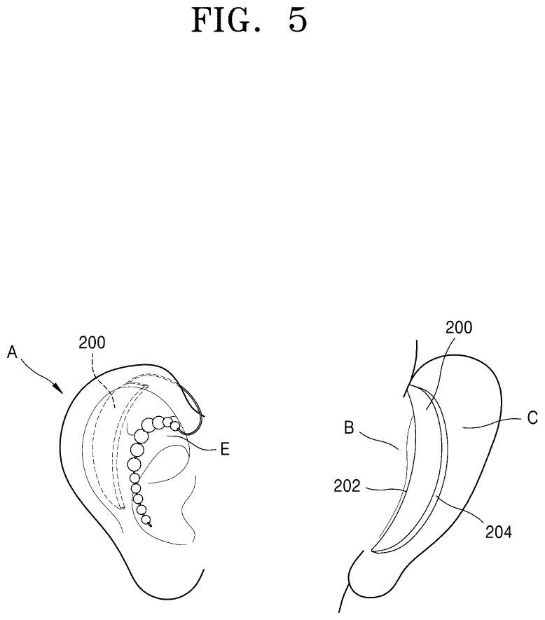

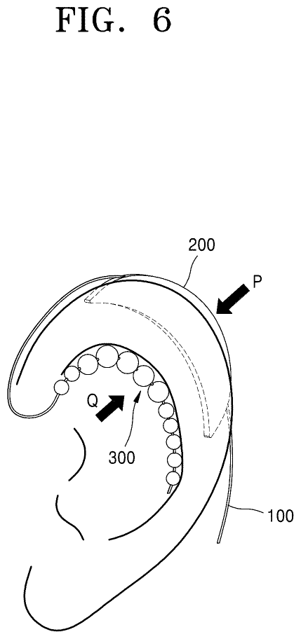

FIG. 3 is a view illustrating body parts around the ear on which the ear correction apparatus 1 will be worn according to an embodiment. FIG. 4 is a view illustrating how the ear correction apparatus 1 is worn according to an embodiment. FIG. 5 is a view illustrating the ear correction apparatus 1 being worn by a user according to an embodiment. FIG. 6 is a view illustrating operational effects of the ear correction apparatus 1 according to an embodiment.

According to an embodiment, the ear correction apparatus 1 may be worn on the ear of a user.

Hereinafter, the ear and body parts around the ear are referred to with reference to FIG. 3 for ease of illustration. Referring to FIG. 3, the ear (part A) and body parts around the ear are named as followed: the scalp (part B) facing the ear, the inner surface (part C) of the ear, an ear root portion (part D) between the ear and the scalp inside the ear, and an outer curved portion (part E) of the ear. The following description is given with the names defined above for ease of illustration.

According to an embodiment, the ear correction apparatus 1 may be worn on the ear as shown in FIG. 5.

The first correction portion 200 is positioned between the part B and the part C. The inner surface 202 of the first correction portion 200 may be in contact with the part B, and the outer surface 203 of the first correction portion 200 may be in contact with the part C. In addition, the support line 210 may be in contact with the part D.

The second correction portion 300 may be positioned on the part E. Because the second correction portion 300 includes the correction balls 310 connected together by the core 100, the second correction portion 300 may deform for stable contact with the part E. In other words, the second correction portion 300 is deformable according to depressions and protrusions of the ear such that the second correction portion 300 may be effectively worn on the ear to provide correction force.

In this case, owing to the curved portion 120 of the core 100, the core 100 may have a shape that hooks onto the ear of a wearer, and thus, the ear correction apparatus 1 may be stably worn on the wearer's ear.

In this case, the first correction portion 200 may be inserted into a space between the part B and the part C. In addition, the support line 210 of the first correction portion 200 may be fixed in position as being supported by the part D.

Thus, as shown by an arrow P in FIG. 6, the first correction portion 200 may support the wearer's ear and maintains the shape of the wearer's ear, thereby providing a shape correction effect on the ear. For example, according to an embodiment, the first correction portion 200 has a crescent shape with a certain thickness such that the first correction portion 200 may entirely support the ear and provide a shape correction effect on the ear.

Additionally, as shown by an arrow Q in FIG. 6, the second correction portion 300 may provide correction force from a front side of the wearer's ear. In addition, according to an embodiment, the second correction portion 300 includes the correction balls 310 and thus deformability, thereby providing an effective correction effect on the wearer's ear. For example, the distance between the first correction portion 200 and some of the correction balls 310 of the second correction portion 300 may be adjusted to concentrate correction force on a portion of the ear located between the first correction portion 200 and some of the correction balls 310.

In addition, according to an embodiment, the correction balls 310 forming the second correction portion 300 have different sizes, and thus, different correction forces may be selectively applied to the ear.

Additionally, in an embodiment, the correction balls 310 forming the second correction portion 300 include the first correction balls 311 and the second correction balls 312, and the first correction balls 311 located at the center portion of the first array line L are relatively large. Thus, the distance (the correction space V) between the second correction portion 300 and the first correction portion 200 may be smaller at the center portion of the first array line L than at the end portions of the first correction portion 200. Thus, correction force at the center portion of the first array line L may be relatively great. This may enable effective correction of the wearer's ear.

The ear correction apparatus 1 of the disclosure may correct the shape of a newborn's ear over a period of several weeks. Thus, various ear deformities, such as cryptotia, Stahl's ear, helical rim deformities, cup ear, and lop ear, may be treated.

The ear correction apparatus 1 of the disclosure may be effectively used for correcting the shape of the ear.

The ear correction apparatus 1 of the disclosure includes the core 100 that may deform when an external force exceeding a certain threshold is applied to the core 100 and may maintain its shape when an external force less than the threshold is applied to the core 100. Thus, the ear correction apparatus 1 may provide an enhanced ear shape correction effect.

The ear correction apparatus 1 of the disclosure includes the first correction portion 200 and the second correction portion 300 that are connected by the core 100, thereby providing an enhanced ear shape correction effect.

The ear correction apparatus 1 of the disclosure includes the second correction portion 300 formed by the correction balls 310, thereby providing an ear shape correction effect tailored to wearers' ears.

The ear correction apparatus 1 of the disclosure may correct the shape of a newborn's ear over a period of several weeks. Thus, various ear deformities, such as cryptotia, Stahl's ear, helical rim deformities, cup ear, and lop ear, may be treated with the ear correction apparatus 1 of the disclosure.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. An ear correction apparatus comprising:
a core having rigidity, ductility, and deformability properties and extending a predetermined length;
a first correction portion connected to the core; and
a second correction portion connected to the core at a position away from the first correction portion,
wherein the first correction portion comprises a support line having a predetermined curvature,
the second correction portion comprises a plurality of correction balls,
the plurality of correction balls are arranged in a row to form a first array line,
the core penetrates the first correction portion in a direction parallel to the support line of the first correction portion, and
the core penetrates the plurality of correction balls along the first array line.

2. The ear correction apparatus of claim 1, wherein the core comprises:
a first line portion penetrating the first correction portion; and
a second line portion penetrating the second correction portion,
wherein at least a portion of the first line portion and at least a portion of the second line portion extend parallel to each other at a predetermined distance from each other,
the support line of the first correction portion and the second correction portion face each other, and
a correction space having a predetermined width is formed between the support line of the first correction portion and the second correction portion.

3. The ear correction apparatus of claim 1, wherein the plurality of correction balls of the second correction portion have different sizes from each other.

4. The ear correction apparatus of claim 1, wherein the plurality of correction balls of the second correction unit comprise first correction balls and second correction balls having a diameter less than a diameter of the first correction balls,
the first correction balls are positioned at a center portion of the first array line, and
the second correction balls are positioned at end portions of the first array line.
